(19) United States
(12) Patent Application Publication
(10) Pub. No.: US 2021/0202096 A1
(43) Pub. Date: Jul. 1, 2021

(54) METHOD AND SYSTEMS FOR SPEECH THERAPY COMPUTER-ASSISTED TRAINING AND REPOSITORY

(71) Applicant: TIKTALK TO ME LTD., Herzliya (IL)

(72) Inventors: Ben-Zion TREGER, Tzur Hadassa (IL); Raphael NASSI, Herzliya (IL)

(21) Appl. No.: 17/058,103

(22) PCT Filed: May 30, 2019
(86) PCT No.: PCT/IL2019/050617

§ 371 Date: Nov. 23, 2020

Related U.S. Application Data
(60) Provisional application No. 62/677,781, filed on May 30, 2018.

(51) Int. Cl. G16H 50/30; G16H 20/30; G10L 15/06; G10L 25/27; G10L 25/51; G06N 20/00

(52) U.S. Cl. CPC G16H 50/30; G16H 20/30; G06N 20/00; G10L 25/27; G10L 25/51; G10L 15/063

(57) ABSTRACT

A computerized system and method of improving a speech therapy process include receiving a signal representing an utterance spoken by the patient, determining a first score reflecting a correlation of the signal and a patient acoustic model, determining a second score reflecting a correlation of the signal and the reference acoustic model, determining a progress metric reflecting a difference between the first score and the second score and responsively providing in real-time an indication of progress to the patient, wherein the indication is presented in at least one of an audio and a visual manner.

### FIELD OF THE INVENTION

The present invention is directed to systems and methods for computer-based patient training, particularly in the field of speech therapy.

### BACKGROUND

The prevalence of speech sound disorder in young children is 8 to 9 percent, and by the first grade, roughly 5 percent of children in the United States have noticeable speech disorders, according to the U.S. National Institute on Deafness and Other Communication Disorders (cited at www.nidcd.nih.gov/health/statistics/statistics-voice-speech-and-language). In addition, speech disorders are prominent in adults after traumatic events such as stroke and physical brain injury. Speech disorders among children include phonetic disorders, articulation disorders, phonemic disorders and developmental apraxia. Among adults, speech difficulties usually result from aphasia, that is, neurological disorders and damage to the speech and language center of the brain. Women and men are equally affected and their number in America reaches about 80,000 new cases per year. About 1 million Americans suffer from aphasia.

Detecting, diagnosing, and treating these disorders can have a profound impact on the quality of people's lives and can help restore normal functioning in adults. The development of an advanced therapeutic platform may have a dramatic impact on the quality of life of a vast population worldwide.

### SUMMARY

Embodiments of the present invention provide systems and methods for speech therapy computer-assisted training. In some embodiments, a computing system is provided including at least one processor and at least one memory communicatively coupled to the at least one processor, the memory comprising computer-readable instructions that when executed by the at least one processor cause the computing system to implement a method of speech therapy assessment. The method may include: training a first acoustic model according to speech of a patient; training a second acoustic model according to speech of a reference speaker; receiving a signal representing an utterance spoken by the patient; determining a first score reflecting a correlation of the signal and the first speech recognition acoustic model; determining a second score reflecting a correlation of the signal and the second speech recognition acoustic model; determining a progress metric reflecting a relationship between the first score and the second score; and, responsively to the progress metric, providing in real-time an indication of progress to the patient, wherein the indication is presented in at least one of an audio and a visual manner.

In further embodiments, the processor may be configured to determine a third score reflecting a correlation of the signal and an acoustic model trained by recent speech of the patient. The third score may be compared to a threshold to determine existence of an equipment problem.

In further embodiments of the present invention, a computing system may be provided wherein computer-readable instructions, when executed by at least one processor of the computing system cause the computing system to implement a method of modifying a speech therapy protocol, including: creating a custom protocol to guide an interactive, automated, speech therapy session of a patient; monitoring behavior parameters of the patient during the speech therapy session; determining that one or more of the behavior parameters meets a predefined behavior threshold requiring a protocol intervention; and making the protocol intervention, comprising modifying a visual or audio output to the patient. In some embodiments, modifying the visual or audio output may include providing an avatar on a patient display to communicate with the patient.

In further embodiments of the present invention, a computing system may be provided wherein computer-readable instructions, when executed by at least one processor of the computing system cause the computing system to implement a method of improving a speech therapy analytics engine. The method may include: creating an analytics engine comprising a set of rules that correlate multiple sets of speech impairments to protocols of automated speech therapy; applying the set of rules to a set of speech impairments for a given patient to generate a custom protocol of automated speech therapy for the given patient; monitoring a metric of progress of the given patient during one or more speech therapy sessions automated by the custom protocol; and, responsively to the metric of progress, modifying the set of rules of the analytics engine. The rules of the rules engine may be generated by a supervised machine learning process, based on a data set of prior protocols and prior therapy outcomes, as applied to former patients. The data set may include data from patients from multiple countries, speaking multiple languages and of multiple ages. The data set may be a first data set, and modifying the set of rules may include generating a new set of rules by a machine learning process, based on a new data set that includes the data of the first data set and the custom protocol and the metric of progress. Creating the analytics engine further may include correlating therapy progress with estimates of patient future progress and providing, given a record of progress for a given patient, an estimated timeframe for the given patient's future progress.

The present invention will be more fully understood from the following detailed description of embodiments thereof.

### BRIEF DESCRIPTION OF DRAWINGS

In the following detailed description of various embodiments, reference is made to the following drawings that form a part thereof, and in which are shown by way of illustration specific embodiments by which the invention may be practiced, wherein:

FIG. 1 shows a schematic, pictorial illustration of a system for conducting speech therapy computer-assisted training, in accordance with an embodiment of the present invention;

FIG. 2 is a schematic, flow diagram of a process for assessing therapy progress, according to an embodiment of the present invention; and

FIG. 3 is a schematic graph of therapy assessment output, according to an embodiment of the present invention.

### DETAILED DESCRIPTION OF THE INVENTION

In the following detailed description of various embodiments, it is understood that other embodiments may be utilized and structural changes may be made without departing from the scope of the present invention.

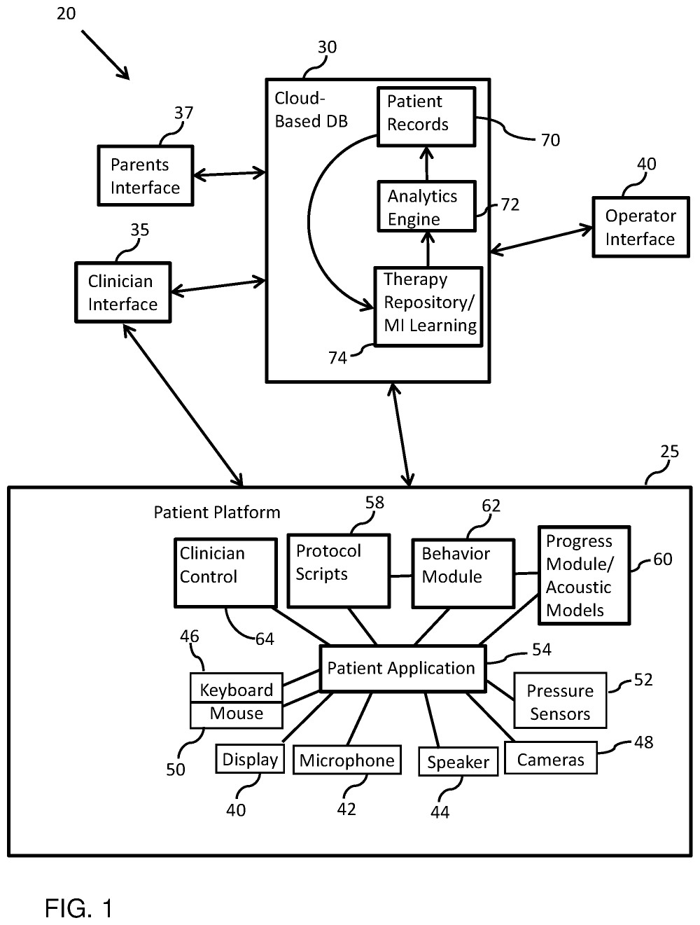

FIG. 1 shows a schematic, pictorial illustration of a system 20 for conducting speech therapy computer-assisted training, in accordance with an embodiment of the present invention. System 20 may include four computer-based systems: a patient platform 25, a cloud-based, back-end database system 30, a clinician interface 35, a parent interface 37, and an operator interface 40.

The patient platform 25 system includes standard computer I/O devices, such as a touch sensitive display 40, a microphone 42, a speaker 44, a keyboard 46 and a mouse 50. In addition, the platform may have one or more cameras 48, as well as air pressure sensors 52, which may be similar to microphones, and which may provide intraoral data related to a patient's pronunciation of sounds. A speech therapy “patient” application 54 runs on the patient platform 25, driving the various output devices and receiving patient responses from the input devices during a patient therapy session. During such a session, the patient application 54 presents games or other interactive activities (collectively referred to hereinbelow as activities), which are intended to train the patient to pronounce certain sounds, words, or phrases correctly. Instructions may be given to the patient visually via the display 40 or audibly via the speaker 44, or a combination of two. The microphone 42 captures the patient's speech and conveys the captured speech signals to the patient application 54, which analyzes the sounds, words, and sentences to identify speech particles, phonemes and words.

The patient application 54 determines the activities to present to the patient, as well as the audio and visual content of each sequential step of those activities, according to a protocol, typically set by one or more protocol scripts 58, which are typically configured by the clinician. The protocol scripts are typically stored in a patient records system 70 of the cloud-based database 30. A protocol is based on general and patient specific clinical guidelines and adapted to a given patient, as described further hereinbelow. Audio content may include sounds, words and sentences presented. Visual content may include objects or text that the patient is expected to visually or auditory recognize and to express verbally. Activities are intended to integrate patient training into an attractive visual and audio experience, which is also typically custom designed according to a patient's specific characteristics, such as age and interests. In some embodiments, the patient application may be a web-based application or plug-in, and the protocol scripts may be code, such as HTML Java applet or Adobe™ Flash (SWF) code.

In embodiments of the present invention, during a patient therapy session, the patient application 54 may send audio, visual, and intraoral signals from the patient to a progress module 60. The progress module may perform one or more assessment tests to determine the patient's progress in response to the protocol-based activities. As described in more detail below, assessments may include analyzing a patient's speech with respect to acoustic models.

Typically, protocol scripts 58 rely on assessments by the progress module 60 to determine how to proceed during a session and from session to session, for example, whether to continue with a given exercise or to continue with a new exercise.

The assessments by the progress module 60 also determine feedback to give a patient. Feedback may include accolades for being successful, or instructions regarding how to correct or improve the pronunciation of the sound or word. Feedback may be visual, audible, tactile or a combination of all three forms. When a patient masters pronunciation of a specific sound or word, the protocol may be configured to automatically progress to the next level of therapy.

Assessments are also communicated to the database system 30, which may be co-located with the patient platform but is more often remotely located and configured to support multiple remote patients and clinicians. The therapy data system may store the assessments with the patient records system 70.

The patient application 54 may also pass speech, as well as other I/O device data, to a behavior module 62, which may identify whether a patient's behavior during a session indicates a need to modify the protocol of the session. The behavior module 62 may operate as a decision engine, with rules that may be generated by a machine learning process. The machine learning process is typically performed by the database system 30, and may include, for example, structured machine learning based on clustering algorithms or neural networks.

The behavior module 62 may be trained to identify, for example, a need to intervene in a custom protocol generated for a patient, according to parameters of behavior measured in various ways during a speech therapy session. Parameters may include, for example, a level of patient motion measured by the cameras 48, or a time delay between a prompt by an activity and a patient's response. Rules of the behavior module typically include types of behavior parameters and behavior thresholds, indicating when a given behavior requires a protocol intervention. Upon detecting that a behavior parameter exceeds a given threshold, the behavior module may indicate to the patient application 54 the type of intervention required, typically by modifying visual and/or audio aspects of the therapy session activity.

If, for example, a patient's ability to repeat words correctly drops from a rate of two trials per word to an average of five trials per word, the behavior module 62 may signal the patient application 54 to reduce a level of quality required for the patient's pronunciation to prevent patient frustration from increasing. If a patient requires too much time to identify objects on the display, that is, a patient's response delay surpasses a threshold set in the behavior module, the behavior module 62 may signal the patient application to increase the size of objects displayed. The behavior module 62 may also receive and analyze video from cameras 48, and may determine, for example, that a patient's movement indicates restlessness. The behavior module may be set to respond to a restlessness indication by changing an activity currently presented by the patient application.

The behavior module 62 may be based on an AI algorithm configured by generalizing patient traits, such as a patient's age, to determine how and when to change a protocol. For example, the behavior module 62 may determine after only one minute a need to modify visual or audio output for a child, based on behavior of the child, whereas the behavior module would delay such a modification (or, “intervention”) for a longer period for an adult patient. One purpose of the behavior module is to prevent a patient from being frustrated to an extent that the patient stops focusing or stops a session altogether. The behavior module 62 may determine that a child, for example, needs an entertaining break for a few minutes. Alternatively, the behavior module may determine a need to pop up a clinician video clip that explains and reminds the patient how to correctly pronounce a sound that is problematic.

The behavior module 62 may also determine from certain behavior patterns that a clinician needs to be contacted to intervene in a session. The patient platform 25 may be configured to send an alert to a clinician through several mechanisms, such as a video call or message. The alert mechanism enables a clinician to monitor multiple patients simultaneously, intervening in sessions as needed.

A clinician control module 64 of the patient platform 25 allows the clinician, working from the clinician interface 35 to communicate remotely with a patient, through the audio and/or video output of the patient platform. In addition, the clinician control module may allow a clinician to take over direct control of the patient application, either editing or circumventing the protocol script for a current session. The clinician interface 35 may be configured to operate from either a desktop computer or a mobile device. A clinician may decide to operate the remote communications either on a pre-scheduled basis, as part of a pre-planned protocol, or on an ad hoc basis. A clinician may also use the clinician interface 35 to observe a patient's session without interacting with a patient. When the patient is a minor, the parents interface 37 may be provided to allow the patient's parent or guardian to track the patient's progress through the treatment cycle. This interface also serves as a communication platform between the clinician and the parent for the purpose of occasional updates, billing issues, questions and answers, etc.

The clinician interface 35 also allows a clinician to interact with the database 30.

In embodiments of the present invention, the database system 30 is configured to enable a clinician to register new patients, entering patient trait data into the patient records system. Patient trait data may include traits such as age and interests, as well as aspects of the patient's speech impediments. The clinician may also enter initial protocol scripts for a patient, a process that may be facilitated by a multimedia editing tool. Alternatively, based on the patient data, an analytics engine 72 of the therapy data system may determine a suitable set of protocols scripts 58 for a patient, as well as suitable behavior rules for the behavior module 62.

Processing by the analytics engine 72 may rely on a rules system, whose rules may be generated by a machine learning process, based on data in the therapy repository 74. Data in the therapy repository 74 typically includes data from previous and/or external (possibly global) patient cases, including patient records, protocols applied to those patients, and assessment results. The machine learning process determines protocols that are more successful for certain classifications of patients and speech impediments, and creates appropriate rules for the analytics engine.

Initial protocols and behavior rules are stored with the patient's records in the patient records system 70, and may be edited by the clinician. During a subsequent therapy session, the database system 30 also tracks a patient's progress, as described above, adding assessment data, as well as other tracking information, such as clinician-patient interaction, in the records system 70. In some embodiments, audio and/or video recordings of patient sessions may also be stored in the records system 70. As a patient progresses from session to session, the clinician can review the patient's progress from the patient's records, and may continue to make changes to the protocols and/or generate protocols or protocol recommendations from the analytics engine 72.

As described above, the database system 30 may be configured to support multiple remote patients and clinicians. Access to multiple clinicians and/or patients may be controlled by an operator through an operator interface 40. Security measures may be implemented to provide an operator with access to records without patient identifying information to maintain patient confidentiality. An operator may manage the therapy repository, transferring patient records (typically without identifying information) to the repository to improve the quality of data for the machine learning process and thereby improve the analytics engine 72.

The process of improving the analytics engine 72 can thus be seen as being a cyclical process. The analytics engine is first generated, typically by a machine learning process that extracts from patient records correlations of patient traits (age, language, etc.), speech disorders, and applied protocols, with progress metrics. This process generates rules that correlate speech impairments and patient traits to recommended protocols for automated speech therapy. The recommend rules are applied to a new patient case to create a custom protocol for the new patient, given the new patient's specific traits and speech impairments. The patient then participates in sessions based on the custom protocol, and the patient's progress is monitored. A metric of the patient's progress is determined, and depending on the level of the progress, the rules of the analytics engine may be improved according to the level of success obtained by the custom protocol.

In some embodiments, the therapy data system is a cloud-based computing system. Elements described above as associated with the patient platform, such as the progress and behavior modules, may be operated remotely, for example at the therapy data system itself.

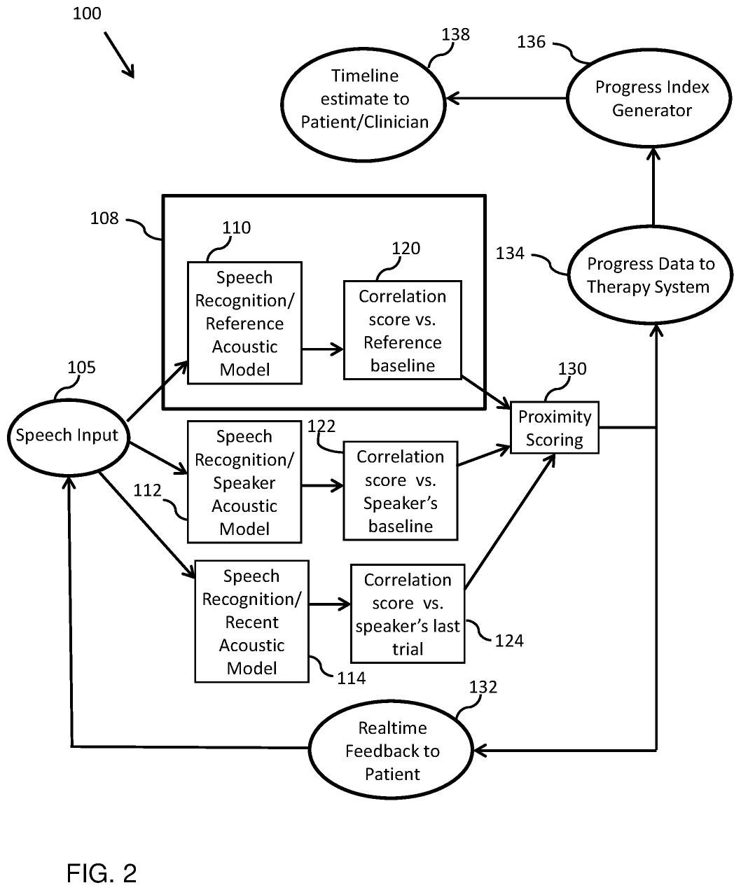

FIG. 2 is a schematic, flow diagram of a process 100 for progress assessment, implemented by the progress module 60, according to an embodiment of the present invention. At steps 110 and 112, speech input 105 from a patient is processed by parallel, typically simultaneous, respective speech recognition processes. The process of step 110 compares the speech input with an acoustic model based on the patient's own patterns of speech (measured at the start of therapy, before any therapy sessions). The process of step 112 similar compares the speech input with an acoustic model based on a reference speaker (or an “ideal” speaker), that is, a speaker with speech patterns that represent a target for the patient's therapy. In some embodiments, speech of the reference speaker may also be the basis for audio output provided to the patient during therapy session. The acoustic models for any given assessment may be general models, typically based on phonemes, and/or may be models of a specific target sound, word or phrase that a patient is expected to utter for the given assessment. By definition, an acoustic model is used in automatic speech recognition to represent the relationship between an audio signal and the phonemes or other linguistic units that make up speech. In embodiments of the present invention, progress of a patient is indicated by the respective correlations of a patient's utterances and the two acoustic models described above.

The output data of steps 110 and 112 are processed, at respective steps 120 and 122, to provide correlations scores. Step 120 provides a score correlating the patient's utterances throughout a therapy session with the patient's acoustic models. Step 112 correlates the patient's utterances with the reference speaker acoustic model at the step. The two scores are compared at a step 130, to provide a “proximity score”, which may be a score of one or more dimensions. In some embodiments, the proximity score may be generated by a machine learning algorithm, based on human expert evaluation of phoneme similarity, i.e., correlation. A perfect correlation with the patient's acoustic model may be normalized as 0, on a scale of 0 to 100, while a perfect correlation with the reference speaker acoustic model may be set to 100. Partial correlations with both models may be translated to a score on the scale of 0 to 100.

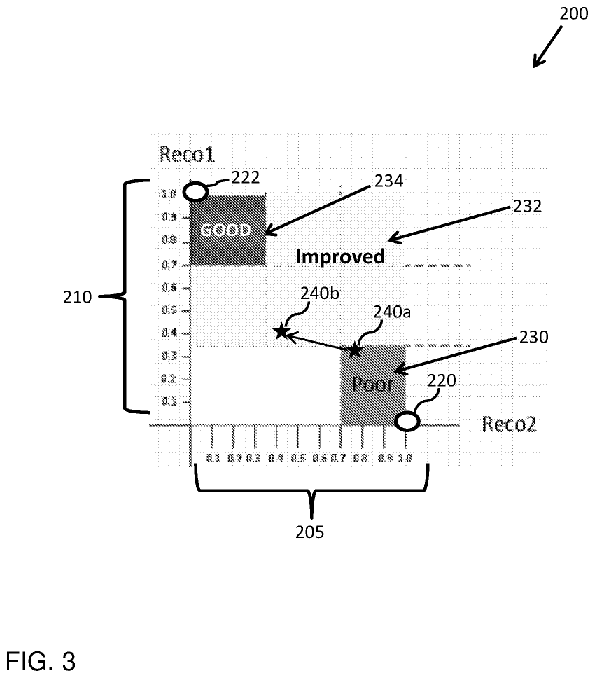

Alternatively, or additionally, the proximity score may be represented on a multi-dimensional (two or more dimension) graph, with axes represented by the correlation scores, as described further below with respect to FIG. 3. Regions of the graph may be divided into sections denoting “poor”, “improved”, and “good” progress.

The proximity score may also have a third component, based on a correlation of a patient's speech, at steps 114 and 124, with an acoustic model based on the patient's speech during a recent (typically the most recent) session. This aspect of the testing can show whether there are immediate issues of changes in the patient's speech that need to be addressed. In addition, the correlation determined at step 124 may indicate whether the equipment may be operating incorrectly. The correlation may be compared with a preset threshold value to indicate an equipment problem.

Based on the proximity score, which represents a metric of the patient progress, the patient application provides a visual and/or audio indication to the patient, at a step 132, representing feedback related to the patient's progress in improving his or her pronunciation. The feedback may be, for example, display of the proximity score, or sounding of a harmonious sound for good progress (e.g., bells), or a non-harmonious sound for poor progress (e.g., honking). Typically the feedback is provided in real time, immediately after the patient has verbally expressed the syllable, word, or phrase expected by the interactive activity.

In some embodiments, the level of progress, as well as encouragement and instructions, may be conveyed to the patient through an “avatar”, that is, an animated character appearing on the display 40 of the patient application 54. The avatar may represent an instructor or the clinician. In further embodiments, the motions and communications of the avatar may be controlled by a live clinician in real time, thereby providing a virtual reality avatar interaction.

In further embodiments, the proximity score (that is, an indicator or the patient's progress) may be also transmitted, at a step 134, to the cloud database system 30. Over the course of one or more therapy sessions, a patient's speech is expected to gradually have less correlation with the patient's original speech recognition acoustic model and more correlation with the reference speaker's speech recognition acoustic model. The patient's progress, maintained at the cloud database system, is available to the patient's clinician.

In addition, the analytics engine 72 of the database system 30 may be configured to apply machine learning processing methods, such as neural networks, to determine patterns of progress appearing across multiple patient progress records, thereby determining a progress timeline estimation model. As a given patient proceeds with therapy sessions, his or her progress may be compared, or correlated with, an index provided by the timeline estimation model, accounting for particular features of the given patient's problems and prior stages of progress. The comparison will provide an estimation of a timeframe for future progress or speed of pronunciation acquisition. In some embodiments, the analytics engine may be supplied with a sufficient number of patient records, to provide a global index. The model may also account for patient characteristics such as language basis, country, age, gender, etc. In further embodiments, the expected timeframe can also be associated with appropriate lessons for achieving target goals within the timeframe provided by the estimate. Consequently, the system will provide an economical and efficient means of designing an individualized course of therapy.

FIG. 3 is a schematic graph 200 of therapy assessment output, according to an embodiment of the present invention. As described above, speech of a patient may be assessed by a dual correlation metric, including a first correlation between an utterance of the patient and an acoustic model of the patient's speech, at the start of therapy (before therapy sessions), and a second correlation between an utterance of the patient and an acoustic model of a reference speaker. Such a metric may be graphed in two dimensions, a first dimension 205 representing the correlation to the patient's acoustic model, and the second dimension 210 representing the correlation to the reference speaker's acoustic model. A point 220, at one extreme point of the graph, represents correlation of the patient's speech to his own acoustic model, at the beginning of therapy. A point 222, at an opposite extreme point of the graph, represents a perfect correlation to the reference speaker acoustic model.

Assuming that the patient progresses, the patient's speech gradually shows less correlation to the patient's original acoustic model and more correlation with the reference speaker acoustic model, that is, a metric of the patient's speech should show an improvement that might be indicated, for example, by point 240a on the graph, and subsequently by a point 240b. Lack of improvement from a given point over time is an indication that a given protocol no longer is effective and that the protocol must be modified. Graph 200 may be divided into three or more sections, to indicate various ranges of improvement. For example, a correlation of 0.7 or more with the patient acoustic model and of 0.35 or less with the reference speaker acoustic model is indicated as a “poor” improvement region 230. A correlation of 0.35 or better with the reference speaker acoustic model is indicated as an “improved” region 232. A correlation of 0.7 or more with the reference acoustic model, and of 0.35 or less with the patient speaker acoustic model, is indicated as a “good” improvement region 234.

It is to be understood that the embodiments described hereinabove are cited by way of example, and that the present invention is not limited to what has been particularly shown and described hereinabove. The rules engines described above may be developed by methods of machine learning such as decision trees or neural networks. The classification of speech scores may include further sub-classifications to distinguish types of difficulties. Additional changes and modifications, which do not depart from the teachings of the present invention, will be evident to those skilled in the art. Computer processing elements described may be distributed processing elements, implemented over wired and/or wireless networks. Such computing systems may furthermore be implemented by multiple alternative and/or cooperative configurations, such as a data center server or a cloud configuration of processers and data repositories. Processing elements of the system may be implemented in digital electronic circuitry, or in computer hardware, firmware, software, or in combinations thereof. Such elements can be implemented as a computer program product, tangibly embodied in an information carrier, such as a non-transient, machine-readable storage device, for execution by, or to control the operation of, data processing apparatus, such as a programmable processor, computer, or deployed to be executed on multiple computers at one site or distributed across multiple sites. Memory storage may also include multiple distributed memory units, including one or more types of storage media.

Communications between systems and devices described above are assumed to be performed by software modules and hardware devices known in the art. Processing elements and memory storage, such as databases, may be implemented so as to include security features, such as authentication processes known in the art.

Method steps associated with the system and process can be rearranged and/or one or more such steps can be omitted to achieve the same, or similar, results to those described herein. The scope of the present invention includes variations and modifications thereof which would occur to persons skilled in the art upon reading the foregoing description and which are not disclosed in the prior art.

1. A computing system comprising at least one processor and at least one memory communicatively coupled to the at least one processor, the memory comprising computer-readable instructions that when executed by the at least one processor cause the computing system to implement a method of speech therapy assessment comprising:
training a first acoustic model according to speech of a patient;
training a second acoustic model according to speech of a reference speaker;
receiving a signal representing an utterance spoken by the patient;
determining a first score reflecting a correlation of the signal and the first speech recognition acoustic model;
determining a second score reflecting a correlation of the signal and the second speech recognition acoustic model;
determining a progress metric reflecting a relationship between the first score and the second score; and,
responsively to the progress metric, providing in real-time an indication of progress to the patient, wherein the indication is presented in at least one of an audio and a visual manner.

2. The computer system of claim 1, wherein the processor is further configured to determine a third score reflecting a correlation of the signal and an acoustic model trained by recent speech of the patient, and to apply the third score to determine an equipment problem.

3. A computing system comprising at least one processor and at least one memory communicatively coupled to the at least one processor, the memory comprising computer-readable instructions that when executed by the at least one processor cause the computing system to implement a method of modifying a speech therapy protocol, comprising:
creating a custom protocol to guide an interactive, automated, speech therapy session of a patient;
monitoring behavior parameters of the patient during the speech therapy session;
determining that one or more of the behavior parameters meets a predefined behavior threshold requiring a protocol intervention; and
making the protocol intervention, comprising modifying a visual or audio output to the patient.

4. The computing system of claim 3, wherein modifying the visual or audio output comprises providing an avatar on a patient display to communicate with the patient.

5. A computing system comprising at least one processor and at least one memory communicatively coupled to the at least one processor, the memory comprising computer-readable instructions that when executed by the at least one processor cause the computing system to implement a method of improving a speech therapy analytics engine comprising:
creating an analytics engine comprising a set of rules that correlate multiple sets of speech impairments to protocols of automated speech therapy;
applying the set of rules to a set of speech impairments for a given patient to generate a custom protocol of automated speech therapy for the given patient;
monitoring a metric of progress of the given patient during one or more speech therapy sessions automated by the custom protocol; and,
responsively to the metric of progress, modifying the set of rules of the analytics engine.

6. The system of claim 5, wherein the rules of the rules engine are generated by a supervised machine learning process, based on a data set of prior protocols and prior therapy outcomes, as applied to former patients.

7. The system of claim 6, wherein the data set includes data from patients from multiple countries, speaking multiple languages and of multiple ages.

8. The system of claim 6, wherein the data set is a first data set, and wherein modifying the set of rules comprises generating a new set of rules by a machine learning process, based on a new data set that includes the data of the first data set and the custom protocol and the metric of progress.

9. The system of claim 5, wherein creating the analytics engine further comprises correlating therapy progress with estimates of patient future progress and providing, given a record of progress for a given patient, an estimated timeframe for the given patient's future progress.
